(19) United States
(12) Patent Application Publication
(10) Pub. No.: US 2025/0160755 A1
(43) Pub. Date: May 22, 2025

(54) PATIENT SUPPORT APPARATUS FOR A MAGNETIC RESONANCE APPARATUS

(71) Applicant: Siemens Healthineers AG, Forchheim (DE)

(72) Inventors: Volker Matschl, Bamberg (DE); Stephan Biber, Erlangen (DE)

(21) Appl. No.: 18/931,812

(22) Filed: Oct. 30, 2024

(30) Foreign Application Priority Data
Nov. 20, 2023 (EP) 23210892.8

(51) Int. Cl. A61B 5/00; A61B 5/055

(52) U.S. Cl. CPC A61B 5/704; A61B 5/055

(57) ABSTRACT

The disclosure relates to a patient support apparatus for a magnetic resonance apparatus, a system including a patient support apparatus and at least one local coil, and a magnetic resonance apparatus. The patient support apparatus includes a patient table having a table surface for positioning a patient. The patient table includes at least one table-side contact field, (e.g., an electrical table-side contact field), for electrically contacting at least one local coil. The at least one table-side contact field is integrated in a flush manner into the table surface.

The present patent document claims the benefit of European Patent Application No. 23210892.8, filed Nov. 20, 2023, which is hereby incorporated by reference in its entirety.

### TECHNICAL FIELD

The disclosure relates to a patient support apparatus for a magnetic resonance apparatus, a system including a patient support apparatus and at least one local coil, and a magnetic resonance apparatus.

### BACKGROUND

In medical technology, imaging by magnetic resonance (MR), (also referred to as magnetic resonance tomography (MRT) or magnetic resonance imaging (MRI)), is distinguished by its high soft-tissue contrast levels. Here, during a magnetic resonance scan with the aid of a magnetic resonance apparatus, radiofrequency excitation pulses are emitted into a patient supported on a patient table, as a result of which magnetic resonance signals are triggered in the patient. The magnetic resonance signals are received by the magnetic resonance apparatus and used for the reconstruction of magnetic resonance mappings.

For receiving the magnetic resonance signals, the magnetic resonance apparatus may have one local coil or several local coils arranged directly on the patient. A local coil may include one or several coil elements (antennas), with which the magnetic resonance signals may be received. The magnetic resonance apparatus may include a signal line for transmitting the received magnetic resonance signals to an evaluation unit of the magnetic resonance apparatus. The signal line may include an interface, (e.g., in the form of a plug-in connection), by which the local coil may be connected to the magnetic resonance apparatus for a magnetic resonance scan. After the magnetic resonance scan, the connection at the interface may be released again in order to remove the local coil. These interfaces may be arranged directly on the patient table.

In order to carry out a magnetic resonance scan as hygienically as possible, it is necessary to be able to clean the patient table, on which the patient is supported during the magnetic resonance scan, in an effective manner.

### SUMMARY AND DESCRIPTION

An object of the present disclosure is to provide a patient table that is easy to clean. The scope of the present disclosure is defined solely by the appended claims and is not affected to any degree by the statements within this summary. The present embodiments may obviate one or more of the drawbacks or limitations in the related art.

Accordingly, a patient support apparatus with a patient table for a magnetic resonance scan is proposed. The patient table has a table surface for supporting a patient, (i.e., the patient may be supported on the table surface). The patient table includes at least one table-side contact field for contacting, in particular electrically and/or optically, at least one local coil. Here, the at least one table-side contact field is integrated in a flush, in particular flat, manner into the table surface.

In particular, the at least one table-side contact field is integrated in a planar manner into the table surface. In particular, the at least one table-side contact field is level with the table surface. In particular, the table surface has no step and/or no elevation and/or no drop in the transition between the at least one table-side contact field and the table surface connected thereto. In particular, the at least one table-side contact field does not project above or below the table surface adjacent thereto. The at least one table-side contact field itself may be smooth and/or level. In particular, the surface of the at least one table-side contact field and the adjacent table surface of the patient table together form a smooth and/or flat and/or level surface overall, in particular without recesses and/or elevations.

Advantageously, such a table surface is easy to clean. By contrast, conventional interfaces, (e.g., plug-in connections), for transmitting magnetic resonance signals between local coils and evaluation units of magnetic resonance apparatuses may have openings for receiving electrical pin contacts. Fluids may enter such openings and may be cleaned only with difficulty.

The at least one table-side contact field may be integrated in a watertight manner into the table surface. This advantageously prevents fluids from penetrating into the interior of the patient table.

The table-side contact field may be suitable for transmitting signals and/or power thereby. The at least one table-side contact field may form a table-side part of an interface for transmitting signals and/or power. For example, magnetic resonance signals received by a local coil may be transmitted via this interface.

The table-side contact field may be configured to transmit electrical and/or optical signals thereby. To this end, the table-side contact field may include electrical and/or optical transmission channels. For example, digital signals may be transmitted via optical transmission channels.

The at least one table-side contact field may include at least one electrical contact, (e.g., galvanic contact), for transmitting signals and/or power. For example, the at least one table-side contact field has a number of electrical contacts corresponding to a number of receive channels or coil elements of a local coil to be connected.

In particular, the at least one table-side contact field has a planar surface, on which several electrical contacts, (e.g., galvanic contacts), are distributed, for example, in the form of a matrix. For example, the several electrical contacts, (e.g., galvanic contacts), may be arranged in rows and/or lines on the at least one table-side contact field.

In particular, the at least one electrical contact is at least one galvanic contact. Advantageously, a signal transmission via galvanic contacts has only minimal losses. The at least one galvanic contact may include an electrically conductive material, (e.g., copper or gold). The at least one galvanic contact may be configured, in a connected state, to (e.g., mechanically) touch a corresponding galvanic contact of a contact field, in particular on the coil side, corresponding to the table-side contact field. As a result, a galvanic connection may advantageously be established between the contacts of the contact fields.

The at least one galvanic contact may be integrated in a planar manner into the surface of the table-side contact field. In particular, the surface of the table-side contact field has no step and/or elevation and/or drop in the transition to the at least one galvanic contact. In particular, the at least one galvanic contact does not project above or below the rest of the surface of the table-side contact field.

The at least one table-side contact field may be configured as a circuit board, in particular, as a printed circuit board (PCB). The at least one contact, (e.g., galvanic contact), of the table-side contact field is applied to, in particular printed onto, a carrier of the circuit board. The material of the carrier may include a composite material, in particular a fiber-reinforced plastic, for example, FR-4. A copper layer, for example, is applied as the contact material. Circuit boards are advantageously particularly easy to manufacture, e.g., in a larger quantity.

The at least one table-side contact field may be freely accessible for an operator of the magnetic resonance apparatus. In particular, the at least one table-side contact field is freely accessible for an operator of the magnetic resonance apparatus from at least one side, in particular from above. This advantageously makes it easy to clean. In particular, the at least one table-side contact field is not covered. In particular, the table-side contact field is accessible in an angular range of at least ±30°, at least ±60°, or ±90° relative to a normal of the surface of the table-side contact field. The normal then corresponds, for example, to a direction directly from or to above.

In particular, the at least one table-side contact field is integrated into the table surface such that a local coil for carrying out a magnetic resonance scan may be laid thereupon. In particular, the local coil may be arranged in a form-fit manner on the table surface.

The patient table may include at least one guide element for, in particular mechanically, guiding, in particular positioning, at least one local coil on the patient table.

The at least one local coil may include at least one corresponding guide element which, during a positioning of the at least one local coil on the patient table, interacts with the at least one guide element of the patient table such that the local coil arrives at a predetermined end position relative to the patient table.

The at least one guide element of the patient table and the at least one guide element of the local coil corresponding thereto may each have a guide surface along which the local coil may slide into the end position.

The patient table may include at least one detector configured to detect a local coil disposed on the table surface (i.e., a “coil presence detector”).

The patient support apparatus may be configured, upon detection of a local coil disposed on the table surface, to switch at least one of the at least one table-side contact field into an active state.

Advantageously, upon detection of a local coil disposed on the table surface, at least one of the at least one table-side contact field may be activated, in particular connected and/or interconnected. The active state may be a state in which a transmission of signals and/or power via the at least one contact field is possible.

Advantageously, in the absence of detection of a local coil disposed on the table surface, the corresponding table-side contact field is deactivated, in particular the electrical contacts thereof are disconnected from the power supply. The deactivated state may be a state in which a transmission of signals and/or power via the at least one contact field is not (or no longer) possible.

As a result, it is advantageously possible to provide that live parts, in particular contacts, do not come into contact with the operator and/or the patient, for example, during the insertion or withdrawal of the local coils. As soon as a local coil is detected, the electrical contacts of the contact field may be electrically connected.

This advantageously dispenses with the need for a mechanical detection device, for example, in the form of a mechanical switch, as such a device may make it more difficult to clean the table surface.

The at least one detector may include at least one capacitive detector, in particular at least one capacitive sensor. A capacitive detector may function on the basis of a change in electrical capacitance of an individual capacitor or a capacitor system. The at least one capacitive detector may include an electrode of such a capacitor or capacitor system. If a local coil is positioned on the patient table, the capacitance of the capacitor or capacitor system advantageously changes such that the presence of the local coil may be registered.

The at least one detector may include at least one inductive detector, in particular at least one inductive sensor. An inductive detector may function on the basis of a change in inductance and/or the quality thereof as a result of a change in position relative to a conductive and/or ferromagnetic part.

The at least one detector may include at least one radio frequency identification (RFID) reader. The RFID reader may be configured to detect an RFID transponder. The RFID transponder is arranged in particular on or in the local coil. This advantageously enables the presence of the local coil to be recognized.

RFID readers may generate a certain electromagnetic field with the aid of a coil and an alternating voltage. If an RFID transponder is brought into this field, power is transmitted to the transponder by way of the field. This enables a microchip of the RFID transponder to be activated in order to decode commands issued from the RFID reader and generate a response, which may in turn be registered by the RFID reader. This advantageously makes it possible not only to detect the presence of a local coil but also to transmit further information from the local coil to the patient support apparatus, such as for example a serial number of the local coil.

The RFID reader may be integrated in the patient table but also in another part of a magnetic resonance apparatus, for example, in a tunnel of the magnetic resonance apparatus.

The at least one detector may include at least one optical detector. For example, the optical detection may take place in a certain spectral range and/or via a glass or plastic optical fiber. It is furthermore conceivable to detect a code, in particular a barcode or a QR code, which is arranged on the local coil to be detected.

The at least one detector may be configured to register properties, in particular resonance properties, of coil elements of a local coil when the local coil is disposed on the table surface. For example, the at least one detector may include a resonance loop. The presence of the local coil may be detected by a resonance increase and/or coupling with a further resonance loop integrated in a local coil.

The at least one detector may be arranged underneath the table surface. This advantageously enables the detector to be better protected against possible damage.

For example, the at least one table-side contact field may be embodied as a circuit board, wherein the electrical, in particular galvanic contacts are arranged on the surface of the circuit board, while inductive and/or capacitive detectors may be fitted in deeper layers of this circuit board so that they cannot be touched, in particular by the operator.

Furthermore, a system is proposed that includes a patient support apparatus as described above and at least one local coil. Here, the at least one local coil includes at least one coil-side contact field, which corresponds to the at least one table-side contact field. The local coil may be a send coil for sending RF signals, a receive coil for receiving magnetic resonance signals, or a send-receive coil for sending RF signals and receiving magnetic resonance signals.

In particular, the at least one coil-side contact field and the at least one table-side contact field each have a contact arrangement that, during positioning of the local coil on the patient table, is made to coincide such that the contacts touch one another and an electrical and/or optical connection is established.

The at least one coil-side contact field and/or the table-side contact field may include at least one electrical spring contact and/or electrical sliding contact. These are advantageously particularly suitable for establishing a reliable contacting. Furthermore, spring contacts and/or sliding contacts are advantageously self-cleaning.

The at least one local coil advantageously includes a rigid region, in which the coil-side contact field is arranged. This rigid region may be an underside of a head coil or a spine coil. The local coil may have no cable for contacting the local coil. Advantageously, a cable-free direct insertion of the local coil on the patient table may be carried out by the contact spring. This advantageously simplifies the workflow of a magnetic resonance scan because the positioning of the local coil on the patient table and the contacting of the local coil may take place simultaneously.

The contacts of the coil-side contact field are advantageously protected against mechanical damage.

The local coil may include a protective mechanism, which exposes the coil-side contact field automatically during positioning of the local coil on the patient table. This may take place in particular mechanically, for example, by a sprung protective surround that is arranged around the coil-side contact field and may be pushed into the interior of the local coil when the local coil is laid onto the patient table. It is further conceivable that the coil-side contact field does not extend out of the coil until a pin is pushed in when the local coil is placed on the table.

Furthermore, a magnetic resonance apparatus with a patient support apparatus as described above is proposed.

### BRIEF DESCRIPTION OF THE DRAWINGS

Further advantages, features, and details of the disclosure are provided in the description below of embodiments, with reference to the drawings. Parts which correspond to one another are provided with the same reference characters in all the drawings.

FIG. 1 depicts an example of a magnetic resonance apparatus with a patient support apparatus and two local coils, (i.e., a head coil and a spine coil).

FIG. 2 depicts an example of a patient support apparatus with two local coils in a disconnected state.

FIG. 3 depicts an example of a patient support apparatus with two local coils in a connected state.

FIG. 4 depicts an example of a top view of a surface of the patient support apparatus.

### DETAILED DESCRIPTION

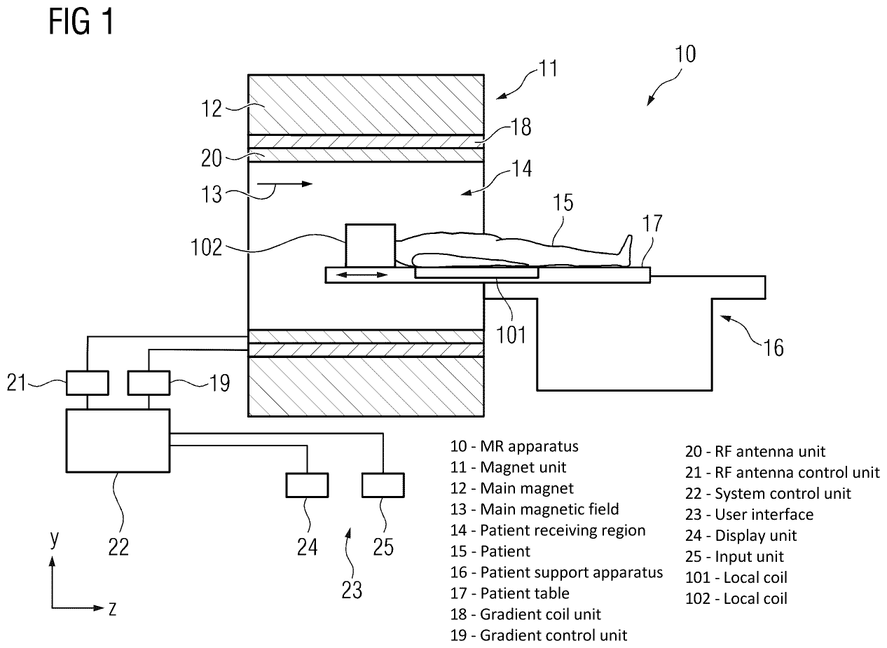

FIG. 1 shows a schematic representation of a magnetic resonance apparatus 10. The magnetic resonance apparatus 10 includes a magnet unit 11, which has a main magnet 12 for generating a strong and in particular temporally constant main magnetic field 13. In addition, the magnetic resonance apparatus 10 includes a patient receiving region 14 for accommodating a patient 15. In the present embodiment, the patient receiving region 14 is embodied cylindrically and is surrounded cylindrically in a circumferential direction by the magnet unit 11. In principle, however, an embodiment of the patient receiving region 14 deviating therefrom is also possible. The patient 15 may be moved by a patient support apparatus 16 of the magnetic resonance apparatus 10 into the patient receiving region 14. For this purpose, the patient support apparatus 16 has a patient table 17 configured to be movable in the z direction within the patient receiving region 14.

The magnet unit 11 also has a gradient coil unit 18 for generating magnetic field gradients that are used for spatial encoding during an imaging process. The gradient coil unit 18 is controlled by a gradient control unit 19 of the magnetic resonance apparatus 10. The magnet unit 11 further includes a radiofrequency antenna unit 20, which is embodied in the present embodiment as a body coil that is fixedly integrated into the magnetic resonance apparatus 10. The radiofrequency antenna unit 20 is controlled by a radiofrequency antenna control unit 21 of the magnetic resonance apparatus 10 and radiates radiofrequency magnetic resonance sequences into an examination space, which is formed by a patient receiving region 14 of the magnetic resonance apparatus 10. By this, an excitation of atomic nuclei by the main magnetic field 13 generated by the main magnet 12 takes place. Through relaxation of the excited atomic nuclei, magnetic resonance signals are generated. The magnetic resonance signals are received by local coils, which are embodied here as a spine coil 101 and as a head coil 102.

For controlling the main magnet 12, the gradient control unit 19 and for controlling the radiofrequency antenna control unit 21, the magnetic resonance apparatus 10 has a system control unit 22. The system control unit 22 centrally controls the magnetic resonance apparatus 10, for example, carrying out a predetermined imaging gradient echo sequence. In addition, the system control unit 22 includes an evaluation unit (not shown in detail) for evaluating the magnetic resonance signals acquired during the magnetic resonance examination. Furthermore, the magnetic resonance apparatus 10 includes a user interface 23 connected to the system control unit 22. Control information, (e.g., imaging parameters and reconstructed magnetic resonance mappings), may be displayed on a display unit 24, (e.g., on at least one monitor), of the user interface 23 for a medical operator. Furthermore, the user interface 23 has an input unit 25 by which information and/or parameters may be input by the medical operator during a scanning procedure.

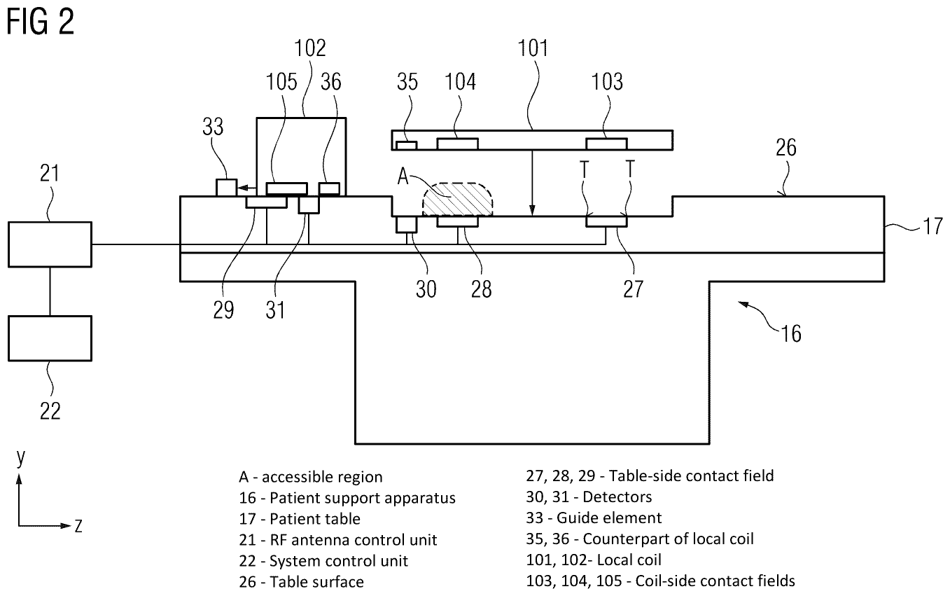

FIG. 2 shows a patient support apparatus 16 with a patient table 17 for use in a magnetic resonance apparatus 10. The patient table has a table surface 26, on which in particular local coils 101, 102 and/or the patient 15 may be arranged. The patient table 17 includes two table-side contact fields 27, 28 for the electrical contacting of a spine coil 101 and a table-side contact field 29 for the electrical contacting of a head coil 102. The table-side contact fields 27, 28, 29 are integrated in a flush, in particular watertight, manner into the table surface. The table-side contact fields 27, 28, 29 and the adjacent table surface 26 together form a smooth overall surface. In particular, there is no edge or suchlike in the transition to the contact fields, in particular at the transition points T, which are drawn in by way of example for the table-side contact field 27. This advantageously makes the contact fields 27, 28, 29 particularly easy to clean.

The contact fields 27, 28, 29 are connected via the radiofrequency antenna control unit 21 to the system control unit 22 of the magnetic resonance apparatus 10. This enables signals and/or information to be transmitted from the system control unit 22 to the contact fields 27, 28, 29 and/or from the contact fields 27, 28, 29 to the system control unit 22.

The table-side contact fields 27, 28, 29 are freely accessible when the local coils 101, 102 are not arranged on the patient table. In particular, the operator may access the contact fields 27, 28, 29 easily from above. Access to the table-side contact fields 27, 28, 29 is not impeded by any covers. By way of example, a freely accessible region A is drawn in for the table-side contact field 28, which contains no elements of the magnetic resonance apparatus 10 which impair accessibility.

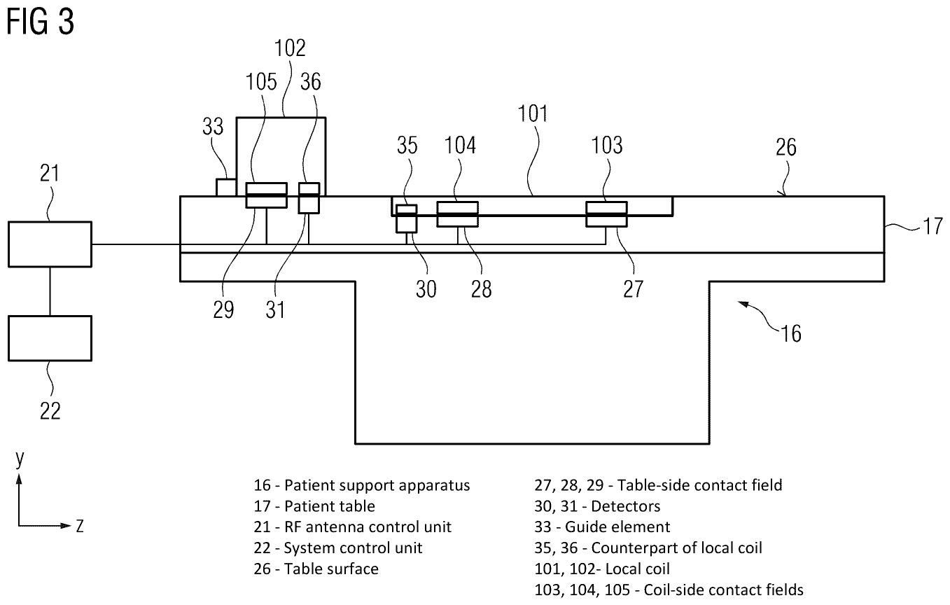

The table surface 26 is shaped to accommodate the spine coil 101. For this purpose, the table surface 26 has a recess which is delimited by the edge 34. Here, this edge 34 also functions as a guide element in order to bring the spine coil 101 into the end position shown in FIG. 3 by moving it in the y direction. In this end position, the table-side contact fields 27, 28 and the coil-side contact fields 103, 104 touch one another. In other words, the table-side contact field 27 comes into contact with the coil-side contact field 103 and the table-side contact field 28 comes into contact with the coil-side contact field 104. In particular, both the table-side contact fields 27, 28 and also the coil-side contact fields 103, 104 in each case have several electrical contacts, (e.g., galvanic contacts), which in the connected state touch one another and may thus transmit signals and/or energy via this interface.

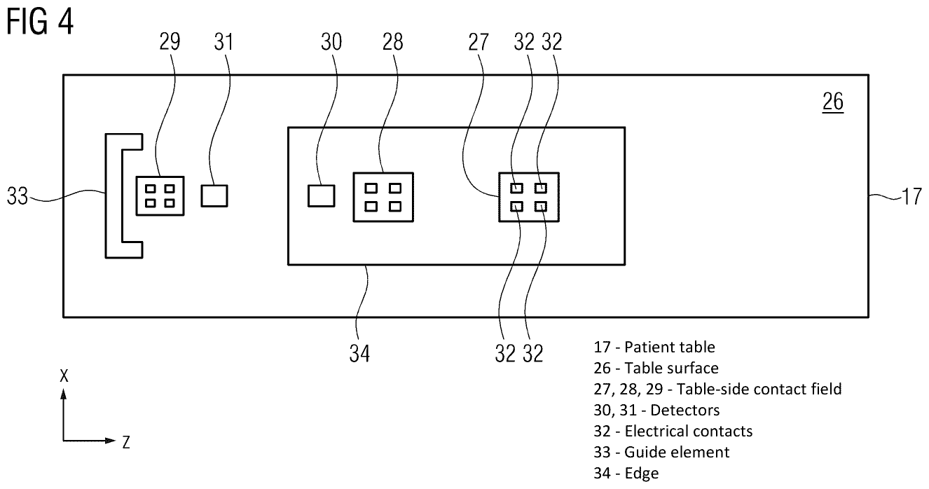

Likewise, the head coil 102 in this example may arrive in the end position shown in FIG. 3 by being moved in the z direction. Here, the movement is supported by the guide element 33 shown in the top view in FIG. 4. In the end position, the table-side contact field 29 comes into contact with the coil-side contact field 105 of the head coil.

The top view also shows electrical contacts 32, (e.g., galvanic contacts), of the table-side contact fields 27, 28, 29 (only provided with reference characters for the contact field 27 for reasons of clarity). It is however also conceivable to arrange one or several optical interfaces on the contact fields for the transmission of optical signals. When the local coils come into their end positions, an electrical connection, (e.g., a galvanic connection), with the corresponding contacts of the coil-side contact fields 103, 104, 105 may be established by these contacts touching one another. In the example shown, each contact field includes four electrical contacts 32, (e.g., galvanic contacts). More or fewer electrical contacts, (e.g., galvanic contacts), may however also be arranged on a contact field. Furthermore, the contact fields may also have a different number of electrical, in particular galvanic, contacts.

The contacts 32 of the table-side contact fields 27, 28, 29 and/or the contacts of the coil-side contact fields 103, 104, 105 may be configured as spring contacts and/or sliding contacts.

The contacts of the coil-side contact fields 103, 104, 105 may be located on the underside of the local coils 101, 102. The contacts of the coil-side contact fields 103, 104, 105 may not be exposed until the local coil 101, 102 is placed or laid onto the planar surface of the patient table 17. This advantageously enables the contacts to be protected against mechanical damage.

For example, the local coil 101, 102 has a sprung protective surround on the underside around the coil-side contact field 103, 104, 105, which is retracted or pressed in when the local coil 101, 102 is laid onto the surface 26 of the patient table 17. It is also conceivable that the local coil 101, 102 has a pin on the underside, which is pressed in when the local coil 101, 102 is laid onto the surface 26 of the patient table 17 and, by a suitable mechanical mechanism, causes the coil-side contact fields 103, 104, 105 and/or the contacts thereof to extend such that they are then exposed.

Furthermore, detectors 30, 31 are arranged in the patient table 17, in particular underneath the table surface 26, which are configured to detect whether a respective associated local coil is disposed or arranged in a predetermined position, in particular in the end positions described above, on the patient table 17. Here, for example, the presence of the spine coil 101 may be detected with the detector 30 and the presence of the head coil 102 with the detector 31.

As long as no presence of the spine coil 101 or the head coil 102 is detected, the respective table-side contact fields 27, 28, 29 are in an inactive state. Such an inactive state may be characterized in that the electrical contacts of the table-side contact fields 27, 28, 29 are disconnected from the power supply. Upon detection of a local coil 101, 102 disposed on the table surface 26, the associated table-side contact field 27, 28, 29 is switched into an active state. If, for example, the detector 30 identifies that the spinal coil column 101 has been laid onto the table surface 26, the contact fields 27, 28 are switched into the active state. Accordingly, the table-side contact field 29 may be switched into the active or inactive state by the detector 31.

In order to carry out such a switchover, a corresponding detection signal may be sent, for example, from the detector 30, 31 to the system control unit 22, which in turn triggers a switching.

For example, the detector 30, 31 is based on a capacitive detection. For this purpose, the detector may include an electrode that interacts capacitively with a counterpart 35, 36 of the local coil 101, 102. An arrangement of the local coil 101, 102 on the patient table 101, 102 may produce a change in capacitance, which is transmitted as a detector signal to the system control unit 22.

According to another embodiment, the detector 30, 31 may also identify the presence of the local coil 101, 102 on the patient table 17 by way of an inductive decoupling.

The table-side contact fields 27, 28, 29 may be configured in the form of a circuit board. Inductive and/or capacitive detector elements, in particular contacts, may be integrated directly into the circuit board, which also contain galvanic contacts 32. The detector elements may be disposed in deeper layers of the circuit board so as to prevent the detector elements from being touched directly by the operator. It may be advantageous for this “coil presence detector,” to be integrated simply into the circuit board, which also contains the galvanic contacts that are then connected.

A further possibility is a detection of an RFID transponder, which is attached in or on the local coil 101, 102. For this purpose, the detector 30, 31 may include an RFID reader. Such an RFID reader may be arranged at another point on the magnetic resonance apparatus 10, such as at the boundary of the patient receiving region 14 or on the housing of the tunnel.

According to another embodiment, the detector 30, 31 includes in each case an optical scanner, which is embodied to identify an optical marking, (e.g., a barcode and/or a QR code), which is applied to the local coil 101, 102. The optical detection may also be detection for example in a certain spectral range, such as via a glass or plastic optical fiber.

It is furthermore conceivable that the detector 30, 31 is configured to identify properties, in particular resonance properties, of coil elements of the local coil 101, 102 when the local coil 101, 102 is disposed on the table surface 26. For example, a simple resonance loop may be arranged in the local coil 101, 102, which couples to a resonance loop in the detector 30, 31 in the patient table 17 so that a resonance increase produces the detection signal.

Finally, it should again be noted that the above detailed description of the patient support apparatus and magnetic resonance apparatus merely sets out embodiments that may be modified by a person skilled in the art in a wide variety of ways without departing from the scope of the disclosure. Furthermore, the use of the indefinite article “a” or “an” does not preclude the possibility that the relevant features may also be present plurally. Similarly, the expression “unit” does not preclude the relevant components including a plurality of interacting subcomponents that may also be spatially distributed, if appropriate.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A patient support apparatus comprising:
a patient table for a magnetic resonance apparatus,
wherein the patient table has a table surface for positioning a patient,
wherein the patient table has at least one table-side contact field for contacting at least one local coil, and
wherein the at least one table-side contact field is integrated in a flush manner into the table surface of the patient table.

2. The patient support apparatus of claim 1, wherein the at least one table-side contact field is integrated in a watertight manner into the table surface.

3. The patient support apparatus of claim 1, wherein the at least one table-side contact field comprises at least one galvanic contact and/or at least one optical interface.

4. The patient support apparatus of claim 1, wherein the at least one table-side contact field comprises a circuit board.

5. The patient support apparatus of claim 4, wherein the circuit board is a printed circuit board.

6. The patient support apparatus of claim 1, wherein the at least one table-side contact field is freely accessible.

7. The patient support apparatus of claim 1, wherein the patient table further comprises at least one guide element for guiding or positioning the at least one local coil.

8. The patient support apparatus of claim 7, wherein the patient table further comprises at least one detector configured to detect a local coil of the at least one local coil that is disposed on the table surface.

9. The patient support apparatus of claim 1, wherein the patient table further comprises at least one detector configured to detect a local coil disposed on the table surface.

10. The patient support apparatus of claim 9, wherein the patient support apparatus configured, upon detection of the local coil disposed on the table surface, to switch a table-side contact field of the at least one table-side contact field into an active state.

11. The patient support apparatus of claim 9, wherein the at least one detector comprises at least one capacitive detector, at least one inductive detector, at least one radio frequency identification (RFID) reader, or a combination thereof.

12. The patient support apparatus of claim 8, wherein the at least one detector comprises at least one optical detector.

13. The patient support apparatus of claim 9, wherein the at least one detector is configured to identify properties of coil elements of the local coil when the local coil is disposed on the table surface.

14. The patient support apparatus of claim 13, wherein the properties comprise resonance properties of the coil elements of the local coil.

15. The patient support apparatus of claim 9, wherein the at least one detector is arranged underneath the table surface.

16. A system comprising:
at least one local coil; and
a patient support apparatus comprising a patient table for a magnetic resonance apparatus,
wherein the patient table has a table surface for positioning a patient,
wherein the patient table has at least one table-side contact field for contacting the at least one local coil,
wherein the at least one table-side contact field is integrated in a flush manner into the table surface of the patient table, and
wherein the at least one local coil comprises at least one coil-side contact field corresponding to a respective table-side contact field of the at least one table-side contact field.

17. The system of claim 16, wherein the at least one local coil comprises a head coil, a spine coil, or a combination thereof.

18. The system of claim 16, wherein the at least one coil-side contact field and/or the at least one table-side contact field comprise at least one spring contact and/or at least one sliding contact.

19. A magnetic resonance apparatus comprising:
a patient support apparatus having a patient table for the magnetic resonance apparatus,
wherein the patient table has a table surface for positioning a patient,
wherein the patient table has at least one table-side contact field for contacting at least one local coil, and
wherein the at least one table-side contact field is integrated in a flush manner into the table surface of the patient table.
